(19) United States
(12) Patent Application Publication
(10) Pub. No.: US 2012/0004303 A1
(43) Pub. Date: Jan. 5, 2012

(54) METHOD OF DISPENSING A TEETHING GEL WITH A METERED DOSE APPLICATOR

(75) Inventors: Jennifer Benson, Levittown, PA (US); Yun Xu, Langhorne, PA (US); Jeffry G. Lavis, Plainsboro, NJ (US)

(73) Assignee: CHURCH & DWIGHT CO., INC., Princeton, NJ (US)

(21) Appl. No.: 12/828,109

(22) Filed: Jun. 30, 2010

(51) Int. Cl. A61K 31/245; A61P 43/00

(52) U.S. Cl. 514/535

(57) ABSTRACT

A method of treating periodontal pain from teething that uses an applicator to dispense a medicament composition, wherein said applicator dispenses a metered dose of a therapeutic amount of said medicament composition.

### FIELD OF THE INVENTION

The present invention provides an improved method for treating periodontal pain, such as periodontal pain caused by teething, using an anesthetic.

### BACKGROUND OF THE INVENTION

Teething is the physiological process of tooth eruption through the gums of the mouth, and typically begins between the ages of four and eight months. Once teething commences, it continues until all twenty childhood teeth are in place at approximately thirty months. Some of the manifestations resulting from the pain of teething include drooling, irritability, sleeping problems and biting on hard objects. The pain is caused by the pressure that erupting teeth place on the periodontal membrane, and periodontal pain caused by the erupting teeth can occur before visually perceptible eruption takes place.

There are presently several remedies for alleviating the periodontal pain caused by teething. These remedies include having the infant suck on a cool object, such as a chilled teething ring. However, sucking on a cool object quickly looses effectiveness as the object warms in the infant's mouth, and once a tooth erupts, parents should avoid using teething rings as the new tooth can puncture them. Other remedies that are used to alleviate the periodontal pain caused by teething include using pain relieving medications, such as acetaminophen and ibuprofen, or homeopathic medications, typically in the form of teething tablets and gels. Using medications such as pain relievers and homeopathic medications has significant drawbacks. For example, acetaminophen and ibuprofen are not recommended for children under two years old unless directed by a physician. In addition, any relief of the teething pain from using these medications is delayed until the drug travels through the bloodstream and takes effect.

An effective remedy to ease the periodontal pain caused by teething is using an anesthetic to control the pain. Anesthesia is a partial or complete loss of sensation or feeling induced by the administration of various substances. There are many different types of anesthesia but they are usually placed into one of three groups. These groups are general anesthesia, local anesthesia, and spinal anesthesia. General anesthetics act primarily on the brain, rendering people both insensible to pain and unconscious. Spinal anesthetics are anesthetics that are injected into the spine. For safety reasons, neither general anesthetics nor spinal anesthetics would be used in treating periodontal pain caused by teething. Local anesthetics, on the other hand, affect only part of the body and the patient remains conscious. Local anesthetics are usually administered through a gel or cream on the surface of the skin or mucosa but can also be injected underneath the skin. Local anesthetics are pharmaceutical materials useful in the relief of many discomforts such as teething, sunburn, pruritus, various dental and surgical procedures, for temporary relief of minor burns, cuts, scratches, nonpoisonous insect bites, poison ivy and other minor skin irritations. They may also be used for postpartum care. In brief, local anesthetics may be employed for diminishing the pain in a restricted area as distinguished from general anesthetics, which are used for eliminating the perception of all stimuli.

When local anesthetics are applied directly to the skin or mucosa they are also referred to as topical anesthetics. Topical anesthetics are absorbed through the skin or mucosa so that they can interact with nerve endings within the dermis. Once the topical anesthetic is absorbed, the topical anesthetic causes a depolarization of sensory nerves within the outer dermis, which temporarily deactivates these nerves. While the anesthetic effect is present, the deactivated sensory nerves do not transmit impulses to the brain. Painful sensations within the anesthetized area are thus temporarily decreased or eliminated.

To treat the periodontal pain caused by teething using a topical anesthetic, a topical anesthetic, typically a gel, is applied onto the affected area in the infant's mouth. Teething gels are applied straight to the gums and provide the quickest pain relief possible. Unlike pain relievers such as acetaminophen and ibuprofen, which can take a half hour or more to work, most teething gels work within a few minutes, and this quick onset of relief from the periodontal pain caused by teething makes teething gels an attractive remedy.

Teething gels currently come in a variety of dispensers. The most common teething gel dispenser is a tube. After cutting off the tip of the tube, the teething gel is generally applied to a finger or a cotton swab. Once the gel is applied to the finger or cotton swab, the gel is then applied to the affected area on an infant's gums. Generally, labels for the teething gels recommend that the dose is a “small amount” or a “small pea-sized amount” which the parent should rub directly to the spot that is hurting. The label for teething gels also suggests that the maximum number of times that the teething gel should be used is up to four times daily, or as directed by a dentist or physician.

One significant drawback to using teething gels to treat the periodontal pain caused by teething is that, while use of a teething gel on the affected area in the infant's mouth will decrease or eliminate the periodontal pain, these topical anesthetic products are difficult to apply in the correct dosage amount to the affected areas. As discussed above, typically caregivers dispense a proscribed amount of topical anesthetic from a tube onto their finger or another object for rubbing onto the infants gums. Because the suggested dosage amounts are relative, i.e. “small” or “small pea-shaped”, the amount of teething gel applied may vary from one caregiver to another. For example, one caregiver's interpretation of “small” may be significantly different from another caregiver's interpretation. In addition, many households have both parents working, so teething gels may also be applied by a third-party caretaker of the infant. Many parents desire that the dosage amount given to their infant remains consistent, regardless of the person applying the teething gel.

In addition to the variance found in applying teething gels from a tube, many teething gels have a four-dose-a-day maximum limit. Many parents want to provide the maximum relief possible within those four doses; however, most anesthetics are limited in the amount that can be applied in order to avoid the risk of overdosing. These concerns of overdosing are applicable in teething gels.

Although teething gels are convenient, safe, and work well to relieve pain on contact, there are certain health concerns that should not be overlooked when using these gels. A condition known as methemoglobinemia has been associated with uses of certain types of teething gels. Methemoglobinemia is when there is too much hemoglobin in a person's red blood cells. According to the FDA, this complication tends to only occur when the medication is used incorrectly, either from too large of a dose or if used for a longer period of time than recommended. By using a tube to dispense teething gel, a caregiver is not able to ascertain how much anesthetic has been applied and how much anesthetic should be applied. Providing less anesthesia in order to alleviate concerns of overdosing would only provide less relief from the pain caused by teething. Instead, what is needed is a method of providing a consistent therapeutic amount of teething gel that would provide the maximum relief from the pain caused by teething.

Thus, what is desired is a method that uses an infant teething gel applicator arrangement that manages the distribution of topical anesthetics over long periods of time, so as to provide consistent dosage amounts that minimize medicinal intake while providing maximum relief of the periodontal pain in the infant. Use of such an applicator would give parents and caregivers peace of mind with the knowledge that they are both providing adequate relief from the periodontal pain in the infants without comprising safety by giving too much anesthetic.

### SUMMARY OF THE INVENTION

The objective of this invention is to provide an improved method for treating the periodontal pain caused by teething, wherein a composition for treating periodontal pain is stored in a container providing a metered dose of a therapeutic amount of the composition when activated, and applying the metered dose onto an area affected by the periodontal pain. In addition to treating periodontal pain caused by teething, the present invention could also be used to treat periodontal pain caused by other sources, such as adult toothache, mouth sores, and/or cold sores, so the container used in the invention could also deliver metered doses for adults.

### BRIEF DESCRIPTION OF THE DRAWINGS

FIG. 1 is a cross-sectional view of a suitable pump assembly for dispensing a medication according to one embodiment of the present invention. Such a pump assembly is manufactured by Yonwoo Co., Ltd. and is available for purchase in the United States.

### DETAILED DESCRIPTION OF THE INVENTION

The objective of this invention is to develop an improved method for treating periodontal pain caused by teething. Current methods for treating periodontal pain typically consist of using a teething gel that is sold in a tube, where the tip is snipped off the tube by a caretaker of an infant in order to ‘open’ the tube which is then sealed with a cap. The caretaker then applies, according to the directions on the package label, a ‘small amount’ of the teething gel onto a fingertip or a cotton swab, and the small amount of teething gel is then applied to the area in the infant's mouth affected by periodontal pain.

However, according to a study on the current methods of using teething remedies, most of the respondents in the study do not use teething remedies as directed on the package. There is a tendency to use less teething gel than directed by the package label (38% of users) while 26% of the users dosed more teething gel than directed. However, by not following the package label, caretakers risk not providing sufficient relief from periodontal pain caused by teething if they apply less teething gel than directed, or they risk causing side effects if they apply more teething gel than directed.

In order to overcome the deficiencies of the prior art methods of using a medication to treat the periodontal pain caused by teething, the present invention provides a method for treating the periodontal pain caused by teething wherein a composition for treating periodontal pain is stored in an applicator providing a metered dose of a therapeutic amount of a teething composition when activated, and applying the metered dose onto an area affected by the periodontal pain. A metered dose applicator is a device used to administer a defined dose of medication. The applicator in the present invention delivers a metered dose each time the applicator is activated, which means that a unit dose, or ‘one dose’, is consistently delivered whenever the container is activated. The applicator should consistently deliver (though occasional outliers may occur) a metered dose within 0-20% of the desired dosage weight, or more preferably, 0-10%, and even more preferably, 0-5%. Thus, for the medication composition described in Example 2, the desired dosage weight is between 0.05-0.30 grams, or more preferably, 0.10-0.20 grams, or even more preferably, 0.12-0.16. For the composition in Example 2, the more preferable desired dosage weight is around 0.15 grams, thus the applicator should consistently deliver a dose that weighs between 0.12 grams and 0.18 grams, or more preferably between 0.135 grams and 0.165 grams, or even more preferably between 0.1425 grams and 0.1575 grams. Oftentimes, an applicator needs to be primed before the applicator can deliver metered doses. Such doses delivered prior to priming are not considered in the variance calculations. Devices which minimize or can avoid the need for priming are particularly preferred. In one embodiment of the present invention, it is demonstrated that the metered dose applicator can be filled with an optimal amount of the teething gel to reduce or even eliminate the need to prime the metered dose applicator.

In addition to treating periodontal pain caused by teething, the present invention could also be used to treat periodontal pain caused by other sources, such as adult toothache, mouth sores, and/or cold sores.

Consistent metered delivery of the medication in the present invention is particularly useful in administering pharmaceutical compositions, such as topical anesthetics like teething gels, which may have side effects at higher dosing levels. The fact that the present invention is intended for topical administration directly to the site of action would also require less amounts of the pharmaceutical additive to be used than would a systemic administration of a pharmaceutical. In accordance with the methods of the invention, the medication used to treat periodontal pain may be a teething gel, which is directly administered to the gums of a teething child. Teething gels typically contain a topical anesthetic to provide relief from periodontal pain. Topical anesthetics included in such a teething gel may be any known anesthetic used to treat teething pain, such as benzocaine, pramoxine, dibucaine, diclonine, lidocaine, mepivacaine, prilocalne, procaine, tetracaine, menthol, camphor and dimethisoquin their pharmaceutically acceptable salts and combinations thereof. It can also be a naturally derived ingredient such as clove oil or eugenol.

Numerous anesthetics, such as the ones listed above, can be used in the present invention and will be apparent to one of ordinary skill in the art. One embodiment of the present invention is benzocaine as an active ingredient of the medication. Benzocaine is known to be an effective topical anesthetic that has an almost immediate onset of pain-relieving action. It is preferred that the anesthetic be thickened by gelling to provide metered doses of the anesthetic as a gelled or otherwise thickened matrix. Such materials facilitate contact of the topical anesthetic with the skin, thereby enhancing the effect thereof.

The anesthetic may be thickened or made viscous in one of several ways, such as adding polyethylene glycol to the composition. A benzocaine ointment thickened with polyethylene glycol has been shown to have an onset of pain-relieving perception within 15 seconds when applied to oral mucosa. A concentration of 7.5% benzocaine disposed in a gelled matrix is available commercially, and is typically used to alleviate teething pain in infants. This material may be advantageously utilized in the present invention. Other topical anesthetics such as lidocaine, procaine, xylocaine, menthol, camphor, clove oil, eugenol and the like may be similarly employed, with or without a thickened matrix, and in various therapeutically effective concentrations.

In another embodiment of the present invention, a flavor-enhancing composition, such as sweeteners or flavors, may be added to teething gel. Oftentimes, medicaments have a bitter or unpleasant off-note taste that adversely affects the overall flavor of the product. If an infant does not like the taste of the medicament, then any relief from the periodontal pain afforded by the anesthetic will be offset by the discomfort of having an off-note taste in the infant's mouth. Sweeteners can be added to the teething gel composition to improve the taste of the medicament. Such sweeteners are known in the art and include, among others, ammonium glycyrrhizate, licorice glycyrrhizates, citrus aurantium, alapyridaine, alapyridaine (N-(1-carboxyethyl)-6-(hydroxymethyppyridinium-3-ol) inner salt, miraculin, curculin, strogin, mabinlin, gymnemic acid, cynarin, glupyridaine, pyridinium-betain compounds, neotame, thaumatin, neohesperidin dihydrochalcone, chlorogenic acid, tagatose, trehalose, maltol, ethyl maltol, quercetin, vanilla extract (e.g., in ethyl alcohol), vanilla oleoresin, vanillin, sugar beet extract (alcoholic extract), sugarcane leaf essence (alcoholic extract), compounds that respond to G-protein coupled receptors (T2Rs and T1Rs), or a combination of at least two of the foregoing.

Flavors can also be added to the medication composition to improve the taste of the medicament. Flavors are known in the art and that can be used in the present invention include those artificial and natural flavors known in the art, for example synthetic flavor oils, natural flavoring aromatics and/or oils, oleoresins, extracts derived from plants, leaves, flowers, fruits, and the like, and combinations comprising at least one of the foregoing flavors. Nonlimiting representative flavors include oils such as spearmint oil, cinnamon oil, oil of wintergreen (methyl salicylate), peppermint oil, clove oil, bay oil, anise oil, eucalyptus oil, thyme oil, cedar leaf oil, oil of nutmeg, allspice, oil of sage, mace, oil of bitter almonds, cassia oil, and citrus oils including lemon, orange, lime, grapefruit, vanilla, fruit essences, including apple, pear, peach, grape, strawberry, raspberry, blackberry, cherry, plum, pineapple, apricot, banana, melon, tropical fruit, mango, mangosteen, pomegranate, papaya, honey lemon, and the like, or a combination of at least two of the foregoing ingredients. Specific flavors are mints such as peppermint, spearmint, artificial vanilla, cinnamon derivatives, and various fruit flavors.

Additives that are generally recognized as safe (GRAS) by FDA can also be included in the medication in effective amounts such as plasticizers or softeners such as lanolin, palmitic acid, oleic acid, stearic acid, sodium stearate, potassium stearate, glyceryl triacetate, glyceryl lecithin, glyceryl monostearate, propylene glycol monostearate, acetylated monoglyceride, glycerine, and the like, to obtain a variety of desirable textures and consistency properties. Waxes, for example, natural and synthetic waxes, hydrogenated vegetable oils, petroleum waxes such as polyurethane waxes, polyethylene waxes, paraffin waxes, microcrystalline waxes, fatty waxes, sorbitan monostearate, tallow, propylene glycol, and the like can also be incorporated into the gum base to obtain a variety of desirable textures and consistency properties. These additives can also improve upon the proper consistency to provide a consistent amount of medication in each metered dose These additives are generally used in amounts of up to about 30 wt % of a medication such as teething gel, specifically about 3 wt % to about 20 wt % of the medication.

Coloring can be used in amounts effective to produce a desired color for the medication. Suitable coloring agents include natural food colors and dyes suitable for food and drug applications. Suitable colors may include annatto extract, bixin, norbixin, astaxanthin, dehydrated beets (beet powder), beetroot red/betanin, ultramarine blue, canthaxanthin, cryptoxanthin, rubixanthin, violanxanthin, rhodoxanthin, caramel, beta-apo-8′-carotenal, beta-carotene, alpha carotene, gamma carotene, ethyl ester of beta-apo-8 carotenal, flavoxanthin, lutein, cochineal extract, carmine, carmoisine/azorubine, sodium copper chlorophyllin, chlorophyll, grape color extract, grape skin extract (enocianina), anthocyanins, fruit juice, vegetable juice, FD&C blue #1, FD&C green #3, FD&C red #40, and FD&C yellow #5 tartrazine.

While the anesthetic formulations described in the present invention is a gel, suitable formulations for topical administration also include other semi-liquid preparations suitable for penetration through the skin to the site of where treatment is required. Other examples of semi-liquid preparations include, but are not limited to creams, ointment or paste. The pharmaceutical ingredients are in general those commonly used and generally recognized by person skilled in the art of pharmaceutical formulation.

Creams, ointments or pastes are semi-solid formulations. They may be made by mixing the pharmaceutically acceptable salts in finely-divided or powdered form, alone or in solution or suspension in an aqueous or non-aqueous fluid, with the aid of suitable machinery, with a greasy or non-greasy base. The base may contain hydrocarbons. Examples of the hydrocarbons include, but are not limited to, hard, soft, or liquid paraffin, glycerol, beeswax, a metallic soap, a mucilage, an oil of natural origin (such as almond, corn, arachis, castor or olive oil), wool fat or its derivative, and/or a fatty acid (such as stearic acid or oleic acid). The formulation may also contain a surface active agent, such as anionic, cationic or non-ionic surfactant. Examples of the surfactants include, but are not limited to, sorbitan esters or polyoxyethylene derivatives thereof (such as polyoxyethylene fatty acid esters), and carboxypolymethylene derivatives thereof (such as carbopol). Suspending agents such as natural gums, cellulose derivatives inorganic materials such as silicaceous silicas, and other ingredients such as lanolin, may also be included. For ointment, polyethylene glycol 540, polyethylene glycol 3350, and propyl glycol may also be used to mixed with the pharmaceutical compound.

The amount of medication delivered per dose by the applicator depends on the type of medication, composition of medication and the rheology of the medication used in the applicator. A person skilled in the art would know, without undue experimentation, the correct dosage amount required for a given medication (see Example 3) and would optimize the formula and rheology or flow property of the medication such that the desired dosage can be delivered consistently. With the proper dosage amount for a given medication, an applicator is chosen that consistently provides the proper dosage amount, or metered dose, of medication per activation.

To produce metered doses according to the present invention, one embodiment of a structure of the metered dose applicator that can be used is an airless type pump (“airless pumps”) to dispense the teething gel. Airless pumps are known in the art, i.e., U.S. Pat. No. 7,367,476, and have been developed for a wide range of applications including dispensing personal care products, such as skin creams, skin lotions, toothpaste, and hair gels, as well as food sauces and the like; however, airless pumps have not been used to deliver metered doses of an anesthetic for periodontal pain. It has been found that airless type dispensing systems are especially efficient in providing a consistent amount of metered doses of a product and can be used in the present invention. Airless pumps also allow dispensing higher viscosity products, such as creams or gels, as opposed to the commonly used pumps, which use dip tubes. In addition to providing metered doses, airless type dispensing systems minimize contact of the medicament with air. Because many anesthetics tend to deteriorate faster when placed in contact with air, it is desired to reduce contact of anesthetics with air. In typical dispensing pump applications, air is allowed to enter the container via a venting path in order to equalize the pressure inside the pack as product is dispensed. Were this not the case, the container would progressively collapse or, in the case of rigid containers, the increasing vacuum in the container would exceed the ability of the dispensing pump to draw product out of the container.

Airless type dispensing systems typically have two methods of dispensing the product, either by using a collapsible bag type design or by using a follower piston-type design. With the collapsible bag type design, a collapsing bag is attached to the dispensing pump, which progressively collapses as the contents are removed. In the follower piston-type design, a rigid container, usually cylindrical or oval in form, has a follower piston that progressively reduces the container volume as product is drawn out by the dispensing pump.

An embodiment of the present invention uses an airless pump with a follower piston to dispense teething gel. While one embodiment of the dispenser used to dispense the teething gel in metered doses is shown here in great detail, it will be apparent to those skilled in the relevant art that other dispensers can be used to dispense teething gel in metered doses.

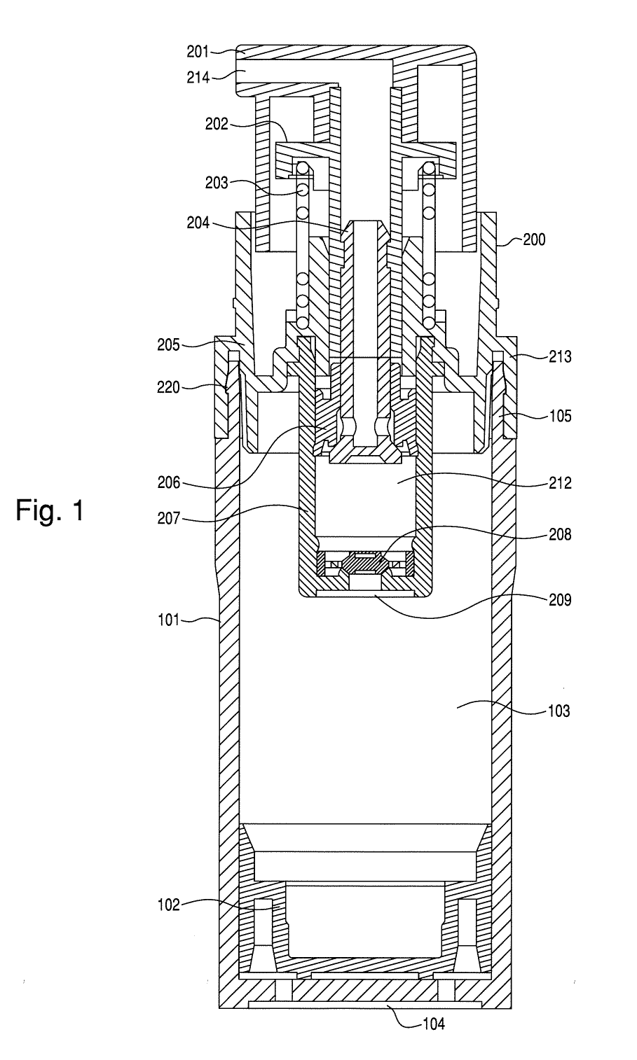

According to one embodiment of the present invention, an airless pump assembly is illustrated in FIG. 1. As shown, the airless pump applicator includes a container 101 for storing teething gel, a follower piston 102 received in the container 101, and a pump 200 for pumping fluid from the container 101. FIG. 1 shows a cross-sectional elevation with the follower piston 102 at the bottom of the container 101. When virtually all of the teething gel in the container 101 is dispensed, the follower piston will be near the top of container 101. It should be noted that directional terms, such as “up”, “down”, “top”, “bottom”, “left” and “right”, will be solely used for the convenience of the reader in order to aid in the reader's understanding of the illustrated embodiments, and that the use of these directional terms in no way limits the illustrated features to a specific orientation. The airless pump assembly will be described with reference to a follower piston type system, but it should be realized that selected features from the assembly can be adapted for use with other types of pumping systems, such as with a collapsible bag type airless dispenser pump.

With reference to FIG. 1, the follower piston 102 is slidably received inside a cavity 103 in the container 101, and the follower piston 102 has upper and lower seal members (not shown) that seal against the container 101. As teething gel is dispensed from the container 101, a slight vacuum is formed, and consequently, the follower piston 102 slides up the cavity 103 to reduce the effective size of the cavity 103. At the base 104, the container 101 has one or more openings (not shown) that vent the container 101 in order to prevent a vacuum from forming between the underside of the follower piston 102 and the base 104 of the container 101 as the follower piston 102 moves progressively upwards during dispensing of the teething gel. In the illustrated embodiment, the container 101 as well as other components have a generally cylindrical shape, but it should be appreciated that these components can be shaped differently in other embodiments.

FIG. 1 also shows the pump assembly in which pump 200 is secured to the container 101 through a snap fit type connection. Nevertheless, it should be appreciated that the pump 200 can be secured to the container 101 in other manners. As can be seen in FIG. 1, the pump body 205 has a shoulder wall 213 and between the outer wall of the pump body and the shoulder wall 213, is a gap. The outer wall of container 101 contains a neck 105 that may include one or more ridges 220 that snap into corresponding grooves in the shoulder wall 213. The neck 105 is inserted into the gap between the shoulder wall 213 and the outer wall of the pump body 205. The pump 200 includes a pump body 205 that is secured to the container 101, an inlet check valve member 208 that controls the flow of fluid into the pump 200, a stem 202 in which a pump piston rod 204 is slidably disposed, an actuator 201 for dispensing the teething gel, and a return spring 203. At one end, the pump body 200 defines an inlet port 209 through which teething gel is received from the container 32.

The pump 200 in the illustrated embodiment is a manually operated by pressing on the pump head 201, but it should be appreciated that the pump 200 in other embodiments can be automatically actuated. After the pump head 201 is pushed down, the spring 203 causes the piston 204 as well as the pump head 201 to return to an extended position. During the upstroke, a vacuum is created in the pump cavity 212. The vacuum formed in the pump cavity 212 causes the inlet check valve member 208 to open, thereby providing a wide through path for the teething gel from the container 101 to enter into the pump cavity 212. On the down or dispensing stroke of the pump 200, the inlet check valve member 208 shuts to prevent the gel in the pump cavity 212 from being pushed back into the cavity 103 of container 101. The teething gel is dispensed via the head nozzle 214. Additional fluid can be dispensed by pressing and releasing the pump head 201 in the manner as described above.

### Example 1

Prior art teething gels typically have a composition as seen in Table 1.

TABLE 1
Prior art composition of a teething gel

| Chemical Name | % Level |
|---|---|
| Polyethylene Glycol 400 | 67.4% |
| Polyethylene Glycol 3350 | 16.87% |
| Sodium Saccharin | 0.75% |
| Sorbitol | 1% |
| Glycerin | 5.25% |
| Sorbic Acid | 0.1% |
| Benzocaine | 7.5% |
| Flavor | 1% |
| Ammonium Glycyrrhizate | 0% |
| FD&C Red No. 40 | 0.0013% |
| Water | 0.1287% |

While the prior art teething gel was effective in treating periodontal pain, it did not have the proper viscosity in order for a metered dose applicator, such as an airless pump, to provide consistent and aesthetically acceptable metered doses of the teething gel.

In order for a metered dose applicator to provide consistent metered doses of a teething gel, a composition for teething gel for use in the present invention is seen in Table 2.

TABLE 2
Composition of teething gel that can
be used in the present invention

| Chemical Name | % Level |
|---|---|
| Polyethylene Glycol 400 | 60.03% |
| Polyethylene Glycol 3350 | 22.99% |
| Sodium Saccharin | 0.75% |
| Sorbitol | 1% |
| Glycerin | 5.25% |
| Sorbic Acid | 0.1% |
| Benzocaine | 7.5% |
| Flavor | 1% |
| Ammonium Glycyrrhizate | 1.25% |
| FD&C Red No. 40 | 0.0013% |
| Water | 0.1287% |

The above composition described in Table 2 results in a pink, homogeneous gel upon discharging of the pump. This composition, when applied to the gums of the infant experiencing periodontal pain from teething, anesthetizes pain sensors. This provides the immediate pain relief to the affected area.

Furthermore, when the above composition described in Table 2 is used in an airless pump, a consistent dosage amount can be delivered per metered dose (see Example 3).

### Example 2

In order to determine the proper dosage amount of the teething gel composition described in Table 2 in each metered dose, a study was done to determine the number of grams typically dispensed when product users dispense ‘a dose’ of teething gel using the prior art method.

Qualified respondents were asked to dispense the amount of teething gel they would typically use for one dose of baby teething gel. The amount dispensed was weighed to the nearest hundredth of a gram.

The mean dose of teething gel was found to be 0.27 grams; however, the median dose weight of 0.15 grams determined in the study was found to be more representative of a typical dose weight based on the skew of the data. According to the study, a metered dose of teething gel contains about 0.10-0.20 grams of teething gel, or more specifically, about 0.12-0.16 grams of teething gel.

While a metered dose was determined to be around 0.10-0.20 grams of teething gel, it will be appreciated by those skilled in the art that the dosage amount in each metered dose depends on the composition of the teething gel (i.e., the amount of anesthetic in the teething gel) and modifications may be made to the dosage amount in each metered dose without departing from the invention in its broader aspects and as set forth in the claims.

### Example 3

An airless pump made according to the specification described above to deliver a metered dose of teething gel. For this particular airless pump system, the target fill weight of the product in the container was set at 6.4 grams. An example of the dosage grams dispensed in each metered dose using an airless pump can be seen in Tables 3.

TABLE 3
Grams of product dispensed per stroke using an airless pump.

| No. of | |
|---|---|
| Stroke | Grams |
| 1 | 0.00 |
| 2 | 0.00 |
| 3 | 0.08 |
| 4 | 0.14 |
| 5 | 0.13 |
| 6 | 0.13 |
| 7 | 0.13 |
| 8 | 0.13 |
| 9 | 0.13 |
| 10 | 0.13 |
| 11 | 0.13 |
| 12 | 0.14 |
| 13 | 0.13 |
| 14 | 0.13 |
| 15 | 0.14 |
| 16 | 0.13 |
| 17 | 0.13 |
| 18 | 0.14 |
| 19 | 0.13 |
| 20 | 0.13 |
| 21 | 0.13 |
| 22 | 0.13 |
| 23 | 0.14 |
| 24 | 0.13 |
| 25 | 0.13 |
| 26 | 0.14 |
| 27 | 0.13 |
| 28 | 0.13 |
| 29 | 0.13 |
| 30 | 0.13 |
| 31 | 0.14 |
| 32 | 0.13 |
| 33 | 0.13 |
| 34 | 0.13 |
| 35 | 0.13 |
| 36 | 0.13 |
| 37 | 0.13 |
| 38 | 0.13 |
| 39 | 0.13 |
| 40 | 0.13 |
| 41 | 0.13 |
| 42 | 0.13 |
| 43 | 0.13 |
| 44 | 0.12 |
| 45 | 0.00 |
| 46 | 0.04 |
| 47 | 0.03 |
| 48 | 0.04 |

As can be seen from Table 3, once an airless pump is primed in the first three strokes, the airless pump dispenser can consistently deliver a dosage amount between 0.12-0.14 grams per stroke. Also, as Table 3 demonstrates, the airless pump dispenser used in this example can deliver approximately 40 metered doses before the residual teething gel can no longer be pumped out due to the construction of the pump system.

While a particular embodiment of the composition used for the treatment of periodontal pain and pump for dispensing same of the invention has been shown and described, it will be appreciated by those skilled in the art that changes and modifications may be made thereto without departing from the invention in its broader aspects and as set forth in the following claims.

1. (canceled)

2. (canceled)

3. (canceled)

4. (canceled)

5. (canceled)

6. A method of treating an affected area experiencing periodontal pain caused by teething comprising:
a) dispensing at least one consistent metered dose from an applicator capable of dispensing multiple and consistent metered doses of a therapeutic amount of a medicament composition, wherein said medicament composition is in a semi-liquid preparation form; and
b) applying said at least one consistent metered dose to said affected area experiencing periodontal pain caused by teething.

7. (canceled)

8. The method of claim 6 wherein said medicament is a composition containing a topical anesthetic.

9. The method of claim 8 wherein said topical anesthetic is benzocaine, clove oil, Eugenol, pramoxine, dibucaine, diclonine, lidocaine, mepivacaine, prilocalne, procaine, tetracaine, menthol, camphor or dimethisoquin and their pharmaceutically acceptable salts and combinations thereof.

10. The method of claim 8 wherein said topical anesthetic is benzocaine.

11. The method of claim 10 wherein said composition is comprised of 5 to 30% benzocaine.

12. The method of claim 10 wherein said composition comprises about 5- to 1-2% benzocaine.

13. The method of claim 6 wherein said composition comprising:
5-20% of a topical anesthetic,
50-80% of polyethylene glycol, and
0-20% of glycerin.

14. The method of claim 13 wherein said topical anesthetic is benzocaine.

15. (canceled)

16. The method of claim 6, wherein said applicator dispenses multiple said doses of said composition.

17. The method of claim 16 wherein said applicator is an airless type pump.

18. The method of claim 16, wherein said metered doses weigh between 0.10-0.20 grams.

19. The method of claim 16, wherein said metered doses weigh between 0.12-0.16 grams.

20. The method of claim 16, wherein said metered doses, after priming of an applicator if necessary, are within 0-20% of a target dosage weight.
